(19) United States
(12) Patent Application Publication
(10) Pub. No.: US 2007/0049954 A1
(43) Pub. Date: Mar. 1, 2007

(54) Esophageal and intestinal coupling devices

(76) Inventors: Michael G. Caty, Amherst, NY (US); Stanley T. Lau, Clarence Center, NY (US)

Correspondence Address: HODGSON RUSS LLP, ONE M & T PLAZA, SUITE 2000, BUFFALO, NY, 14203-2391, US

(21) Appl. No.: 11/510,874

(22) Filed: Aug. 24, 2006

Related U.S. Application Data
(60) Provisional application No. 60/711,203, filed on Aug. 25, 2005.

(51) Int. Cl. A61B 17/08

(52) U.S. Cl. 606/153

(57) ABSTRACT

A surgical apparatus and method for joining esophageal, intestinal, or other tubular tissue segments comprises a delivery tube and a delivery docking member each carrying a coupling member seated at its distal end for delivering the coupling members to a terminal location in a respective tissue segment. An actuating rod received through the delivery tube and threadably mated with the docking arm adjusts the delivery tube relative to the docking arm to push the coupling members together causing them to fasten to one another and join the tissue segments. The bodies of the coupling members are preferably formed of biodissolvable material to degrade as the tissue segments grow together.

### CROSS-REFERENCE TO RELATED APPLICATIONS

The present application claims benefit of U.S. Provisional Patent Application No. 60/711,203 filed Aug. 25, 2005, the entire disclosure of which is hereby incorporated herein by reference.

### FIELD OF THE INVENTION

The invention relates generally to the field of surgical devices and procedures, and more particularly to a coupling device for joining tubular tissue segments, such as esophageal or intestinal segments, and to a surgical tool and method for delivering the coupling device within a patient.

### BACKGROUND OF THE INVENTION

Esophageal atresia is a serious birth defect that occurs in approximately 1 of every 3000 live births. The esophagus of children born with this condition is segmented into separate upper and lower esophageal portions (commonly referred to as esophageal pouches or sacs) and does not form a continuous passageway to the stomach. In most forms of esophageal atresia, the upper esophageal sac begins to fill with mucus and saliva shortly after birth. Consequently, excessive drooling, choking, and coughing are symptomatic of esophageal atresia. An infant afflicted with esophageal atresia will expel whatever he or she is fed, thereby preventing digestion and absorption of orally administered foods.

There are several types of esophageal atresia. In one type, the upper and lower esophageal sacs are not attached to the trachea. In the most common type, the upper esophageal portion ends as a blind sac, whereas the lower esophageal portion is connected to the trachea by a narrow canal at a point just above the tracheal bifurcation. In yet another type, the narrow canal between the trachea and the distal portion of the esophagus forms a ligamentous cord. In rare instances, both portions of the esophagus actually open into the trachea.

Currently, operative repair requires a thoracotomy with a hand-sewn anastamosis between the two portions of the esophagus. A thoracotomy, or incision through the chest wall, has a number of both immediate and long-term sequelae. This incision is associated with a significant amount of post-operative pain and contributes to post-operative pulmonary impairment and an increased length of hospital stay. In addition, there are a number of long-term implications of this incision, including rib deformity, scoliosis, and shoulder dysfunction. And as cosmetic outcome has become progressively more important, minimally invasive techniques have come to play a more significant role in today's modern surgical world.

Thoracoscopic methods of repairing esophageal atresia are being used by some medical centers. This technique uses the technology of traditional laparoscopic surgery. Laparoscopic instruments are used to sew the two ends of the esophagus together, a technique that is far more demanding than a traditional hand-sewn anastamosis in open surgery (through a thoracotomy incision). A surgeon must have considerable technical skill to perform this operation.

Other techniques for treatment of esophageal atresia are based on the fact that for a period of about three months after birth, the esophageal sacs spontaneously undergo a period of rapid growth toward each other, and these techniques seek to encourage this process. One such technique is described in U.S. Pat. No. 3,986,493 entitled “Electromagnetic Bougienage Method.” According to this technique, an infant is prepared for a subsequent anastomosis without tension. In particular, an external annular electromagnet is used to intermittently magnetize bougies located within each esophageal sac. Over a period of time, the intermittent force created between the bougies causes the lengthening of the atretic segments. Once the atretic segments are sufficiently lengthened to allow a primary anastomosis, the magnetic bougies are removed and the esophagus is surgically joined. This technique has several drawbacks. The use of an external electromagnet requires that the infant be placed in a specially constructed bed having a large annular electromagnet. This bed apparatus is expensive and the electromagnet itself can significantly impact the ability of caregivers to nurture the infant. In addition, the use of an external electromagnet requires a subsequent procedure to surgically join the esophageal sacs into a continuous lumen. Surgically joining the esophageal sacs requires great surgical skill, and can present significant operative and post-operative complications. For example, surgical joining can result in the misalignment of the esophagus, and consequently, difficulty swallowing. Additional complications include gastroesophageal reflux, which can lead to ulcers in the lower part of the esophagus.

Another technique taking advantage of the initial rapid growth period involves surgically applying sutures to the opposing ends of the esophageal sacs. The sutures create traction forces to the ends of the esophageal sacs during the rapid growth period, thereby causing further elongation of the esophageal sacs. Ultimately, the sutures cause the esophageal sacs to grow together. A main drawback of using sutures is the need for a significant surgical procedure once the esophageal sacs are sufficiently lengthened by the traction. This procedure involves surgically joining the esophageal sacs, which can result in a number of the complications mentioned above. Moreover, as the sutures draw the esophageal sacs together, the sutures frequently tear out of one or both of the sacs. This requires at least one, and often multiple additional surgeries to re-suture the esophageal sacs.

Yet another technique for achieving a more rapid approximation of the esophageal sacs during the rapid growth period is described in U.S. Patent Application Publication No. 2005/0228412 A1 entitled “Pediatric Atresia Magnets.” This publication describes a medical device that includes an esophageal catheter and a gastric catheter each having a magnetic tip at its distal end. The distal end of the esophageal catheter is passed through the esophagus to the upper esophageal sac, and the distal end of the gastric catheter is passed through a gastrostomy and into the lower esophageal sac. The magnetic forces created by both magnets results in approximation of the esophageal sacs, pressure-induced necrosis establishes a passageway between the esophageal sacs. A stent or stent-graft can be deployed within the established passageway to prevent re-stenosis of the esophagus. During this procedure, the esophageal catheter and a gastric catheter remain in the patient for 5-7 days, if not longer, presenting a risk of infection. Another drawback of this technique is its dependence on fistula formation between the two ends of the esophageal sacs. A high rate of stricture formation and leakage are predictable. To date, this technique has not been accomplished.

In another situation, one of the more technically difficult procedures a surgeon may have to perform is a laparoscopic bowel anastamosis, where two ends of bowel are joined together. There are several methods in use today. A surgeon may choose to sew the two ends of intestine together using standard laparoscopic instruments. This method is technically very demanding, and since the consequences of a mistake are quite severe, only a limited number of surgeons are capable of performing this technique. The surgeon may also try to use a stapling device to join the two ends of bowel. This technique requires the surgeon to properly align the intestine, and he or she must still sew a portion of the bowel (albeit in a more simple fashion). In addition, the intestine must be opened in order to insert the stapling device, risking the chance of spilling stool into the abdomen and leading to infectious complications. The last option that a surgeon may choose is to make a small incision, pull the intestine out of the abdomen, and sew the bowel together in the standard open fashion. Obviously, this requires a larger incision in order to deliver the intestine outside of the abdomen.

### SUMMARY OF THE INVENTION

It is an object of the present invention to provide a simple apparatus and minimally invasive method for correcting esophageal atresia in a technically simple manner that would avoid the necessity of a thoracotomy and the technical difficulties of exposing the esophagus and performing a hand-sewn anastamosis.

It is another object of the present invention to provide a simple apparatus and minimally invasive method for performing an intestinal anastamosis in a technically simple manner

Accordingly, the present invention generally provides an apparatus and a method for joining two tubular segments of living tissue end-to-end to permit formation of a single tubular segment of living tissue. The invention is intended for use in joining esophageal portions, intestinal portions, or other tubular tissue portions. An apparatus formed in accordance with an embodiment of the present invention comprises a delivery tube including a proximal tube end, a distal tube end opposite the proximal tube end, and an external seat near the distal tube end; a delivery docking arm including a proximal arm end, a distal arm end opposite the proximal arm end, an external seat near the distal arm end, and a threaded arm portion near the distal arm end; a first coupling member having a passage therethrough, the first coupling member being mounted on the delivery tube by insertion of the distal tube end into the passage of the first coupling member; a second coupling member having a passage therethrough, the second coupling member being mounted on the delivery docking arm by insertion of the distal arm end into the passage of the second coupling member, wherein the first and second coupling members fasten to one another when pushed together while their respective passages are substantially in axial alignment; and an actuating rod extending through the delivery tube, the actuating rod including a proximal rod end, a distal rod end opposite the proximal rod end, a threaded rod portion near the distal rod end for mating with the threaded arm portion of the delivery docking arm, and an external abutment surface between the threaded rod portion and the proximal rod end, wherein the actuating rod is axially adjustable relative to the delivery docking arm by mating of the threaded rod portion with the threaded arm portion, and the abutment surface of the actuating rod engages the proximal tube end. By axial adjustment of the actuating rod relative to the delivery docking arm, the first and second coupling members are pushed together and caused to fasten to one another with their respective passages in axial alignment.

The above apparatus is used to carry out a surgical method of the present invention in which the first and second coupling members are delivered to respective segments of tubular tissue and urged together to joint the tissue segments, thereby allowing the tissue segments to grow together. According to an embodiment of the invention, the method comprises the steps of providing the first coupling member and the second coupling member as mentioned above; mounting the first coupling member on the delivery tube; mounting the second coupling member on the delivery docking arm; inserting the delivery tube through the first tubular segment of tissue to a location adjacent a distal end of the first tubular segment; inserting the docking arm through the second tubular segment of tissue to a location adjacent a distal end of the second tubular segment; inserting an actuating rod having a threaded rod portion through the delivery tube and mating the threaded rod portion with the threaded arm portion of the delivery docking arm, wherein the actuating rod includes an external abutment surface for engaging a proximal end of the delivery tube; threadably adjusting the actuating rod relative to the delivery docking arm to urge the delivery tube and the delivery docking arm toward one another such that the first and second coupling members are pushed together and fastened to one another, whereby the distal end of the first tubular segment is joined in contact with the distal end of the second tubular segment to form the singular tubular segment; and removing the actuating rod, the delivery docking arm, and the delivery tube from the patient.

Preferably, the bodies of the first and second coupling members are made of a biodissolvable material that degrades as the tissue segments grow together, thus obviating the need for surgical removal of the coupling members.

### BRIEF DESCRIPTION OF THE DRAWINGS

The nature and mode of operation of the present invention will now be more fully described in the following detailed description of the invention taken with the accompanying drawing figures, in which:

FIG. 1 is a side view of a surgical tool formed in accordance with a preferred embodiment of the present invention;

FIG. 2 is a perspective view of a coupling device of the present invention implantable in a patient using the surgical tool shown in FIG. 1;

FIG. 3 is a view generally illustrating a surgical method of implanting the coupling device in accordance with the present invention; and

FIG. 4 is an enlarged view illustrating use of the surgical tool to fasten together first and second members of the coupling device.

### DETAILED DESCRIPTION OF THE INVENTION

The present invention encompasses an apparatus and method for surgically joining a first generally tubular segment of living tissue end-to-end with a second generally tubular segment of living tissue. For purposes of the present description, the invention is described primarily in the context of joining esophageal portions of an infant afflicted with esophageal atresia. However, it will be understood that the present invention is applicable to joining separate segments of other generally tubular organs, for example two intestinal portions.

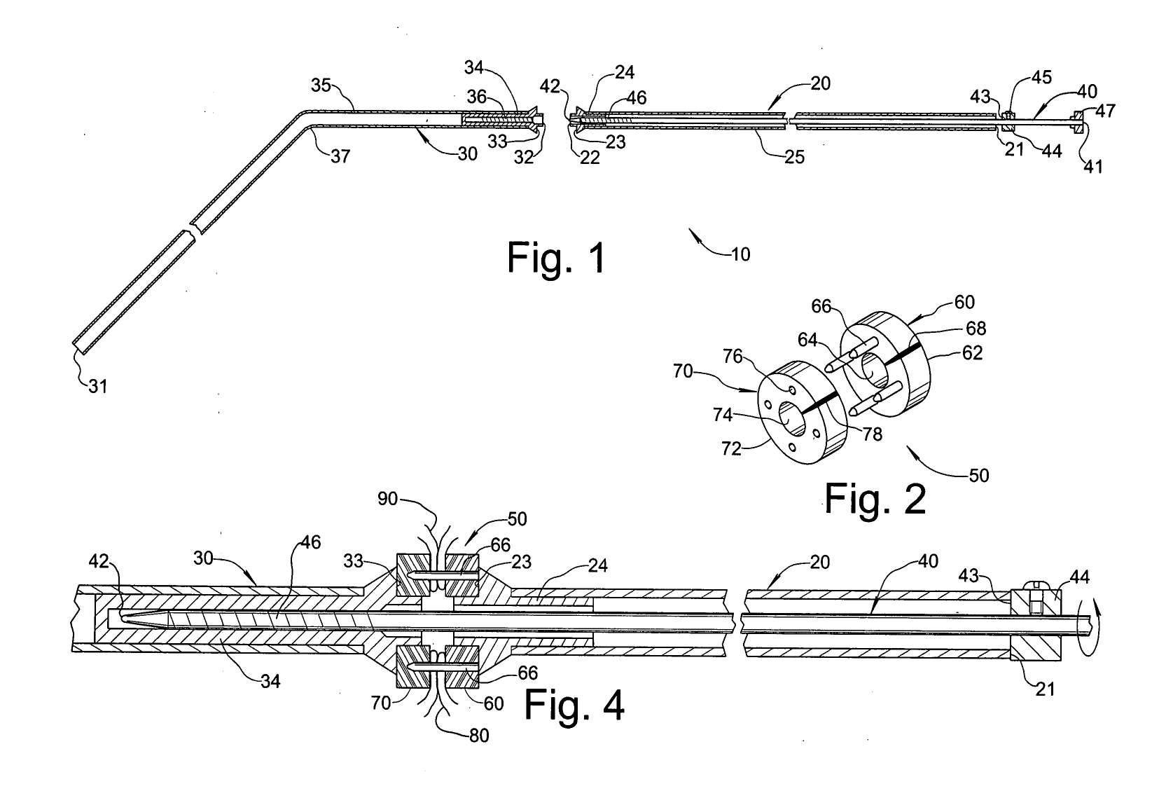

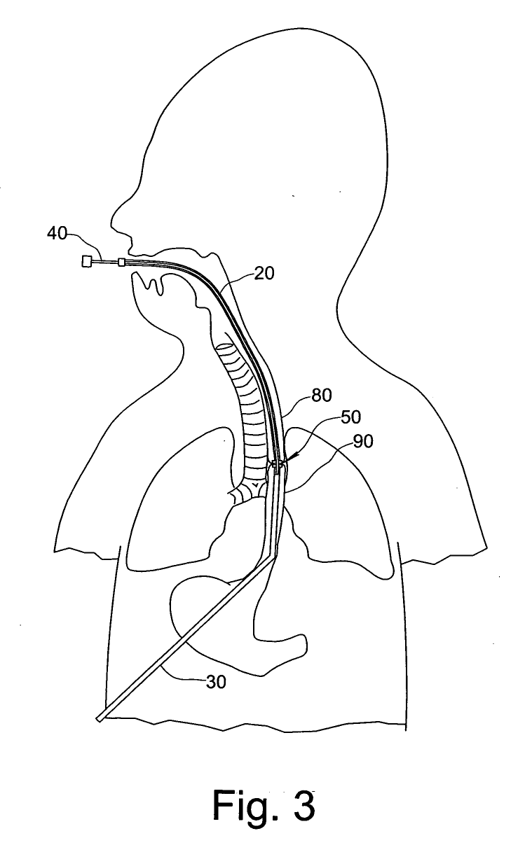

FIG. 1 depicts a surgical tool 10 and FIG. 2 depicts a coupling device 50 which together comprise the apparatus of the present invention. Surgical tool 10 is used to implant coupling device 50 within a patient, as will be described with additional reference to FIGS. 3 and 4.

In the embodiment shown in FIG. 1, surgical tool 10 generally comprises a delivery tube 20, a delivery docking arm 30, and an actuating rod 40. Delivery tube 20 is preferably in the form of an elongated catheter that includes a proximal tube end 21, a distal tube end 22 opposite proximal tube end 21, and an external seat 23 located near distal tube end 22. Delivery tube 20 may be formed as a one-piece unit from a biocompatible polymer, or by coupling a machined insert 24 at an end of a length of tubing 25 as illustrated in FIG. 1. Delivery tube 20 preferably has a smooth outer surface, and may be sized for insertion through an infant's mouth and into the infant's esophagus. In the embodiment shown in FIG. 1, external seat 23 is provided by an annular enlargement on insert 24, but may also be provided by an annular enlargement formed integrally with the rest of delivery tube 20. External seat 23 is preferably reinforced on the side of proximal tube end 21 and configured to provide a stable, flat surface facing distal tube end 22. The length of delivery tube 20 is chosen to allow distal tube end 22 to reach the terminus of an upper esophageal segment or sac while proximal tube end 21 remains outside the patient. A catheter tubing size of 12 french and an external seat diameter of 10 mm are regarded as suitable delivery tube dimensions for esophageal delivery in most infants. It will be understood, however, that a range of sizes may be offered.

Delivery docking arm 30 is preferably an elongated cylindrical member that includes a proximal arm end 31, a distal arm end 32 opposite proximal arm end 31, an external seat 33 near distal arm end 32, and a threaded arm portion 36 near distal arm end 32. Delivery docking arm 30 may be formed as a one-piece unit from a biocompatible polymer, or by coupling a machined insert 34 at an end of a length of tubing or cylindrical rod 35 as shown in FIG. 1. Delivery docking arm 14 preferably has a smooth outer surface, and may be sized for insertion through a gastrostomy and into a lower esophageal segment or sac via the stomach. An obtuse bend 37 is preferably provided about 15 centimeters from distal arm end 32 to facilitate insertion of delivery docking arm 30 within the patient. In the embodiment shown in FIG. 1, external seat 33 is provided by an annular enlargement on insert 34, but may also be provided by an annular enlargement formed integrally with the rest of delivery docking arm 30. External seat 33 is preferably reinforced on the side of proximal arm end 31 and configured to provide a stable, flat surface facing distal arm end 32. In the present embodiment, threaded arm portion 36 is an internally threaded portion that extends approximately from the axial location of external seat 33 to an axial location approximately 2.5 centimeters therefrom in a direction toward proximal arm end 31. The length of docking arm 30 is designed to allow distal arm end 32 to reach the terminus of a lower esophageal segment or sac while enabling a surgeon to manipulate proximal arm end 31. The external diameters of docking arm 30 and its external seat 33 may be chosen to correspond with the dimensions of delivery tube 20 and its external seat 23.

Actuating rod 40 is longer than delivery tube 20 and includes a proximal rod end 41, a distal rod end 42 opposite proximal rod end 41, a threaded rod portion 46 near distal rod end 42 for mating with threaded arm portion 36 of delivery docking arm 30, and an external abutment surface 43 between the threaded rod portion 46 and the proximal rod end 41. Actuating rod 40 is arranged to extend through delivery tube 20 during surgery, and serves as an adjustment member for bringing delivery tube 20 and delivery docking arm 30, and their respective external seats 23 and 33, closer together. As may be understood, abutment surface 43 will engage against proximal tube end 21 as actuating rod 40 is screwed into delivery docking member 30 to thereby urge delivery tube 20 and delivery docking member 30 toward one another. In the depicted embodiment, actuating rod 40 includes an adjustable stop collar 44 for defining abutment surface 43, wherein the stop collar is slidably adjustable along the actuating rod and fixable in a selected axial position by a radial set screw 45. In this manner, the axial position of abutment surface 43 can be adjusted to provide intended cooperation among delivery tube 20, delivery docking member 30, and actuating rod 40. It is noted, however, that abutment surface 43 may be defined by a fixed surface along actuating rod 40, such as a surface of an enlarged head 47 at proximal rod end 41, and stop collar 44 may be omitted without straying from the present invention. Distal rod end 42 is preferably pointed in order to puncture tissue during surgery as will be described below. Actuating rod 40 is preferably manufactured from stainless steel, aluminum, plastic, or a material having similar properties.

Referring now to FIG. 2, coupling device 50 is a two-part device that includes a first coupling member 60 and a second coupling member 70. In the embodiment shown in FIG. 2, first coupling member 60 includes a ring-shaped body 62 having a passage 64 extending axially through body 62. Likewise, second coupling member 70 is shown as including a ring-shaped body 72 having a passage 74 extending axially through body 72. Passages 64 and 74 preferably have the same diameter. Coupling members 60 and 70 are configured to fasten to one another when pushed together while their respective passages 64 and 74 are substantially in axial alignment. For this purpose, one of the coupling members (either first coupling member 60 or second coupling member 70) may include one or more fasteners protruding from the body of such coupling member for engaging the body of the other coupling member. For example, in the illustrated embodiment, first coupling member 60 includes a plurality of pins 66 partially embedded in body 62 to protrude from the body. Second coupling member 70 is shown as including a plurality of corresponding holes 76 in body 72 for respective receipt of pins 66 when coupling members 60, 70 are pushed together. Depending upon the nature of the material forming bodies 62 and 72, and whether pin are pointed at their protruding ends, holes 76 may be unnecessary. Pins 66 may have barbs to prevent separation of coupling members 60, 70. Of course, other fastening configurations are possible. By way of non-limiting example, one or more staples may be partially embedded as fasteners, and the body of the receiving coupling member may be configured with a groove corresponding to the staple(s) that acts as an anvil to fold the protruding staple portions over the body of the receiving coupling member. Other arrangements may be possible wherein fasteners are not embedded, but the bodies 62, 72 are themselves configured to mate and fasten to one another when pushed together. As will be appreciated, the exact mechanism of fastening is not important, what is important is that the coupling members fasten to one another when pushed together. Preferably, the bodies 62 and 72 are provided with respective radiopaque markings 68, 78 that align with one another when passages 64, 74 are in axial alignment. Markings 68, 78 may be used during surgery to locate and properly align coupling members 60, 70. The dimensions of coupling members 60, 70 may be chosen based upon the intended delivery route, such as through the anus, through the mouth, or through a gastrostomy, and based upon the intended destination, such as the esophagus or intestine. The outer diameter of the distal end 22 of delivery tube 20 and the outer diameter of distal end 32 of delivery docking member 30 are selected for slidable insertion into coupling member passages 64 and 74, respectively.

In accordance with the present invention, the bodies 62, 72 of coupling members 60, 70 are made of a biodissolvable material. In the present specification, “biodissolvable material” is intended to include materials that degrade, dissolve, or are absorbed inside a patient's body, for example polyglactin. A particular commercially available biodissolvable material suitable for practicing the present invention is sold by Ethicon (a Johnson & Johnson company) under the trademark VICRYL®.

A surgical method of implanting coupling device 50 to join upper and lower esophageal sacs will now be described with reference to FIGS. 3 and 4, wherein an upper esophageal sac is designated by reference numeral 80 and a lower esophageal sac is designated by reference numeral 90. Apparatus 10 is prepared by mounting first coupling member 60 on distal tube end 22 until it is flush against external seat 23, and mounting second coupling member 70 on distal arm end 32 until it is flush against external seat 33. A small abdominal incision is made to create a gastrostomy (a hole in the stomach) that allows access to lower esophageal sac 90. Delivery docking arm 30 is then passed through the gastrostomy up into lower esophageal sac 90 until second coupling member 70 is at the terminus of the lower sac, and delivery tube 20 is passed through the patient's mouth and down into upper esophageal sac 80 until first coupling member 60 is at the terminus of the upper sac. A surgical imaging device (not shown) may be used to image radiopaque markings 68, 78 on coupling members 60, 70 to assist in locating and aligning the coupling members. Actuating rod 40 is inserted through delivery tube 20 until its threaded rod portion 46 punctures upper sac 80 and lower sac 90, is received into distal arm end 32, and mates with threaded arm portion 36. If the upper and/or lower esophageal portions 80, 90 are not closed sacs, distal rod end 42 may not have to puncture tissue thereof. Once the threaded rod portion 46 is mated with threaded arm portion 36, proximal rod end 41 of actuating rod 40 is rotated in a clockwise direction as indicated in FIG. 4 to advance actuating rod 40 into docking arm 30. As may be understood, abutment surface 43 of actuating rod 40 engages against proximal tube end 21 to force delivery tube 20 and docking arm 30 toward one another. Consequently, the respective external seats 23 and 33 move toward one another, to push coupling members 60 and 70 together with their respective passages 64 and 74 in axial alignment. Coupling members 60 and 70 fasten to one another, thereby joining the terminal ends of upper and lower sacs 80, 90 together in contact with one another. Actuating rod 40 is then unscrewed from docking arm 30 and removed from the patient, and delivery tube 20 and docking arm 30 are removed from the patient, leaving coupling members 60, 70 in place within the patient. If the patient is noted to have a connection between lower sac 90 and the trachea, a surgical clip is delivered by thoracoscopy to divide the connection. A feeding tube is then placed into the stomach through the gastrostomy, allowing the patient to receive post-operative nourishment.

Over time, the upper and lower sacs 80, 90 grow together to form a single tubular segment of tissue and the bodies 62, 72 of coupling members 60, 70 dissolve. Pins 66 are passed in the patient's stool.

Those skilled in the art will realize that a similar procedure may be performed to join two intestinal portions, wherein delivery of one of the coupling members is through the anus rather than the mouth.

As will be appreciated from the foregoing description, the apparatus and method of the present invention provide a surgical procedure for joining esophageal, intestinal, or other tubular tissue segments with a lower degree of operating skill on the part of the surgeon as compared to existing techniques. Moreover, the need for subsequent surgical procedures is eliminated in most cases.

What is claimed is:

1. An apparatus for joining two generally tubular segments of living tissue end-to-end to permit formation of a single generally tubular segment of living tissue, the apparatus comprising:
a delivery tube including a proximal tube end, a distal tube end opposite the proximal tube end, and an external seat near the distal tube end;
a delivery docking arm including a proximal arm end, a distal arm end opposite the proximal arm end, an external seat near the distal arm end, and a threaded arm portion near the distal arm end;
a first coupling member having a passage therethrough, the first coupling member being mounted on the delivery tube by insertion of the distal tube end into the passage of the first coupling member;
a second coupling member having a passage therethrough, the second coupling member being mounted on the delivery docking arm by insertion of the distal arm end into the passage of the second coupling member;
wherein the first and second coupling members fasten to one another when pushed together while their respective passages are substantially in axial alignment; and
an actuating rod extending through the delivery tube, the actuating rod including a proximal rod end, a distal rod end opposite the proximal rod end, a threaded rod portion near the distal rod end for mating with the threaded arm portion of the delivery docking arm, and an external abutment surface between the threaded rod portion and the proximal rod end;
wherein the actuating rod is axially adjustable relative to the delivery docking arm by mating of the threaded rod portion with the threaded arm portion, and the abutment surface of the actuating rod engages the proximal tube end;
whereby the first and second coupling members are pushed together by axial adjustment of the actuating rod relative to the delivery docking arm to cause the first and second coupling members to fasten to one another with their respective passages in axial alignment.

2. The apparatus according to claim 1, wherein the first coupling member and the second coupling member each include a body made of biodissolvable material.

3. The apparatus according to claim 2, wherein the biodissolvable material includes polyglactin.

4. The apparatus according to claim 1, wherein the threaded arm portion of the delivery docking arm is internally threaded, and the threaded rod portion of the actuating rod is externally threaded.

5. The apparatus according to claim 1, wherein the distal end of the actuating rod is pointed for puncturing tissue.

6. The apparatus according to claim 1, wherein the actuating rod includes an enlarged head at the proximal rod end, and the abutment surface of the actuating rod is defined by the enlarged head.

7. The apparatus according to claim 1, wherein the actuating rod includes a stop collar axially adjustable to a selected position along the actuating rod, and the abutment surface of the actuating rod is defined by the stop collar.

8. A method of joining a first generally tubular segment of living tissue end-to-end with a second generally tubular segment of living tissue to form a single generally tubular segment of living tissue, the method comprising the steps of:
providing a first coupling member and a second coupling member each having a passage therethrough, wherein the first and second coupling members fasten to one another when pushed together while their respective passages are substantially in axial alignment;
mounting the first coupling member on a delivery tube;
mounting the second coupling member on a delivery docking arm having a threaded arm portion;
inserting the delivery tube through the first tubular segment to a location adjacent a distal end of the first tubular segment;
inserting the docking arm through the second tubular segment to a location adjacent a distal end of the second tubular segment;
inserting an actuating rod having a threaded rod portion through the delivery tube and mating the threaded rod portion with the threaded arm portion of the delivery docking arm, wherein the actuating rod includes an external abutment surface for engaging a proximal end of the delivery tube;
threadably adjusting the actuating rod relative to the delivery docking arm to urge the delivery tube and the delivery docking arm toward one another such that the first and second coupling members are pushed together and fastened to one another, whereby the distal end of the first tubular segment is joined in contact with the distal end of the second tubular segment to form the singular tubular segment; and
removing the actuating rod, the delivery docking arm, and the delivery tube from the single tubular segment.

9. The method according to claim 8, wherein the first tubular segment and the second tubular segment are esophageal portions.

10. The method according to claim 9, wherein at least one of the esophageal portions is an esophageal sac, and the step of inserting the actuating rod through the delivery tube and mating the threaded rod portion with the threaded arm portion includes puncturing the esophageal sac with the actuating rod.

11. The method according to claim 8, wherein the first tubular segment and the second tubular segment are intestinal portions.

12. The method according to claim 8, wherein the contacting distal ends of the first and second tubular segments grow together.

13. The method according to claim 8, wherein the first coupling member and the second coupling member each include a body made of biodissolvable material.

14. A coupling device for joining two generally tubular segments of living tissue end-to-end to permit formation of a single generally tubular segment of living tissue, the coupling device comprising:
a first coupling member having a body made of biodissolvable material and a passage through the body; and
a second coupling member having a body made of biodissolvable material and a passage through the body;
wherein the first and second coupling members fasten to one another when pushed together while their respective passages are substantially in axial alignment.

15. The coupling device according to claim 14, wherein one of the first and second coupling members includes at least one fastener protruding from the body of such coupling member for engaging the body of the other coupling member.

16. The coupling device according to claim 15, wherein the at least one fastener includes a pin.

17. The coupling device according to claim 15, wherein the at least one fastener includes a staple.

18. The coupling device according to claim 14, wherein the biodissolvable material includes polyglactin.

19. The coupling device according to claim 14, wherein the body of the first coupling member and the body of the second coupling member are each ring-shaped.

20. A surgical tool for use in delivering a coupling device for joining two generally tubular segments of living tissue end-to-end to permit formation of a single generally tubular segment of living tissue, the coupling device including first and second coupling members each having a passage therethrough, the surgical tool comprising:
a delivery tube including a proximal tube end, a distal tube end opposite the proximal tube end, and an external seat near the distal tube end for carrying a first coupling member;
a delivery docking arm including a proximal arm end, a distal arm end opposite the proximal arm end, an external seat near the distal arm end for carrying a second coupling member, and a threaded arm portion near the distal arm end; and
an actuating rod extending completely through the delivery tube, the actuating rod including a proximal rod end, a distal rod end opposite the proximal rod end, a threaded rod portion near the distal rod end for mating with the threaded arm portion of the delivery docking arm, and an external abutment surface between the threaded rod portion and the proximal rod end;
wherein the actuating rod is axially adjustable relative to the delivery docking arm by mating of the threaded rod portion with the threaded arm portion, and the abutment surface of the actuating rod engages the proximal tube end;
whereby the seat of the of the delivery tube and the seat of the delivery docking arm are pushed together by axial adjustment of the actuating rod relative to the delivery docking arm.

21. The surgical tool according to claim 20, wherein the external seat of the delivery tube is an annular enlargement integrally formed with the delivery tube.

22. The surgical tool according to claim 20, wherein the external seat of the delivery docking arm is an annular enlargement integrally formed with the delivery docking arm.

23. The surgical tool according to claim 20, wherein the threaded arm portion of the delivery docking arm is internally threaded, and the threaded rod portion of the actuating rod is externally threaded.

24. The surgical tool according to claim 20, wherein the distal end of the actuating rod is pointed for puncturing tissue.

25. The surgical tool according to claim 20, wherein the actuating rod includes an enlarged head at the proximal rod end, and the abutment surface of the actuating rod is defined by the enlarged head.

26. The surgical tool according to claim 20, wherein the actuating rod includes a stop collar axially adjustable to a selected position along the actuating rod, and the abutment surface of the actuating rod is defined by the stop collar.
